(19) United States
(12) Patent Application Publication
(10) Pub. No.: US 2016/0213599 A1
(43) Pub. Date: Jul. 28, 2016

(54) COSMETIC COMPOSITION COMPRISING A SULFONIC POLYMER, A HYDROPHOBIC POLYSACCHARIDE AND SILICONE SURFACTANT

(71) Applicant: L'OREAL, Paris (FR)

(72) Inventor: Marie DEVIE, Antony (FR)

(73) Assignee: L'OREAL, Paris (FR)

(21) Appl. No.: 14/915,517

(22) PCT Filed: Aug. 14, 2014
(86) PCT No.: PCT/FR2014/052090

§ 371 Date: Feb. 29, 2016

(30) Foreign Application Priority Data
Aug. 30, 2013 (FR) 1358341

(51) Int. Cl. A61K 8/894; A61K 8/81; A61Q 19/00; A61K 8/73

(52) U.S. Cl. CPC A61K 8/894; A61K 8/73; A61K 8/8158; A61K 8/8147; A61Q 19/00; A61K 2800/594; A61K 2800/48

(57) ABSTRACT

The invention relates to a cosmetic composition comprising, in a physiologically acceptable aqueous medium, a polysaccharide modified with hydrophobic chains; a polymer comprising sulfonic groups; a (meth)acrylic acid polymer, which is preferably crosslinked, and/or an oxyethylenated silicone surfactant.

Use for caring for and making up keratin materials.

The present invention relates to compositions, in particular cosmetic compositions, comprising at least one polysaccharide modified with hydrophobic chains, at least one sulfonic polymer and at least one acrylic polymer and/or one oxyethylenated silicone surfactant, and also to the use of these compositions in a process for treating human keratin materials. More particularly, the compositions of the invention are intended for caring for and/or making up keratin materials.

For the purposes of the invention, the term “keratin materials” is intended to denote, for example, the skin, the mucous membranes, the lips, the scalp, the eyelashes, the eyebrows and the hair.

A subject of the present invention is a composition, in particular of the oil-in-water emulsion type, of fluid to viscous consistency, intended for caring for, treating or making up keratin materials such as the skin, the nails, the eyelashes, the eyebrows, the hair or else lips.

This composition may be a foundation, a face powder, an eyeshadow, a concealer, a lip cream, a mascara or a body makeup product, when it is in colored form, or else may be a skin care cream, a conditioner, a shampoo, an anti-sun cream, a skin-coloring cream, or else a dermatological ointment, when it is in non-colored form.

These compositions have varied textures ranging from fluid to solid and generally contain oils and pulverulent materials which are optionally coloring. One of the difficulties encountered by users is that of being able to uniformly spread the composition, for example the foundation, well over the entire surface of the face so as to uniformly distribute the product. Compositions with very thick textures or solid compositions are difficult to spread because of their high viscosities. Compositions with fluid textures are not always suitable for obtaining a uniform makeup result which does not leave visible marks on the skin, in particular because of the poor spreading of said compositions over the entire surface of the face to be made up.

A composition which is too fluid is difficult to apply to keratin materials. Such a composition runs on keratin materials, in particular on the skin, onto which it is applied. Its application to the keratin materials that it is desired to treat lacks precision and therefore makes it relatively unattractive to use.

It is therefore often sought to formulate compositions which have a sufficiently thick texture to enable easy handling of the product, both when taking the composition out of its packaging, and also when applying it to the skin and/or the keratin materials, without however being solid to the point of it no longer being possible to take it up or spread it uniformly.

It is common practice to introduce thickeners into the external phase of emulsions, or else to increase the content of dispersed internal phase so as to obtain the desired viscosity. However, during the industrial production thereof, it is then necessary to have equipment that is sufficiently powerful to allow uniform dispersion of the internal phase in the thickened continuous phase. Such equipment is, however, considerably more expensive.

Moreover, the presence of pulverulent materials such as pigments or fillers, and/or the presence of active agents, has the drawback of destabilizing the compositions, in particular emulsions, and/or of significantly modifying the viscosity of the phase which comprises them.

There is therefore a need for stable cosmetic compositions which have a sufficiently high viscosity to allow satisfactory handling of the product, particularly during application thereof.

The inventors have shown that it is possible to obtain such a composition by combining at least one hydrophobically modified polysaccharide, at least one sulfonic polymer, at least one acrylic polymer and/or one oxyethylenated silicone surfactant.

Surprisingly, the inventors have observed that the compositions exhibit good stability over time, in particular after storage for 2 months at ambient temperature (25° C.) and at 45° C. The compositions do not undergo phase separation and the viscosity does not change or changes little over time.

Furthermore, this composition makes it possible to incorporate active agents and/or fillers: the presence of the fillers and/or of the active agents does not modify the stability.

In addition, the composition according to the invention, when it is applied to keratin materials, and in particular to the skin, does not have a tacky feel; it leaves a soft, velvety, non-greasy finish on the skin.

More specifically, the present invention relates to a composition comprising, in a physiologically acceptable aqueous medium, i) at least one polymer comprising a sulfonic group, ii) at least one hydrophobically modified polysaccharide and iii) at least one (meth)acrylic acid polymer and/or one silicone surfactant chosen from oxyethylenated polydimethylsiloxanes.

According to the invention, the viscosity of the compositions can range from 5 to 100 Poises, preferably from 10 to 50 Poises at 25° C. The composition according to the invention is in particular a cosmetic composition.

The present invention also relates to a non-therapeutic treatment process for caring for or making up keratin materials, comprising the application to said keratin materials of a composition in accordance with the invention.

Advantageously, such a process is intended for caring for or making up the skin.

According to the invention, the term “at least one” is understood to mean “one, two or more”.

### Hydrophobically Modified Polysaccharide

The composition according to the invention comprises at least one hydrophobically modified polysaccharide, preferably a fructan.

Fructans or fructosans are oligosaccharides or polysaccharides comprising a sequence of anhydrofructose units optionally combined with several saccharide residues other than fructose. Fructans may be linear or branched. Fructans may be products obtained directly from a plant or microbial source or alternatively products whose chain length has been modified (increased or decreased) by fractionation, synthesis or hydrolysis, in particular enzymatic. Fructans generally have a degree of polymerization from 2 to about 1000 and preferably from 2 to about 60.

Three groups of fructans are distinguished. The first group corresponds to products whose fructose units are for the most part linked via β-2-1 bonds. These are essentially linear fructans such as inulins.

The second group also corresponds to linear fructoses, but the fructose units are essentially linked via β-2-6 bonds. These products are levans.

The third group corresponds to mixed fructans, i.e. containing β-2-6 and β-2-1 sequences. These are essentially branched fructans, such as graminans.

The fructans preferably used in the compositions according to the invention are inulins. Inulin may be obtained, for example, from chicory, dahlia or Jerusalem artichoke. Preferably, the inulin used in the composition according to the invention is obtained, for example, from chicory.

The fructans, in particular the inulins, used in the compositions according to the invention are hydrophobically modified. In particular, they are obtained by grafting hydrophobic chains onto the hydrophilic backbone of the fructan.

The hydrophobic chains that may be grafted onto the main chain of the fructan may in particular be linear or branched, saturated or unsaturated hydrocarbon-based chains containing from 1 to 50 carbon atoms, such as alkyl, arylalkyl, alkylaryl or alkylene groups; divalent cycloaliphatic groups or organopolysiloxane chains. These hydrocarbon-based or organopolysiloxane chains may in particular comprise one or more ester, amide, urethane, carbamate, thiocarbamate, urea, thiourea and/or sulfonamide functions such as in particular methylenedicyclohexyl and isophorone; or divalent aromatic groups such as phenylene.

In particular, the fructan in particular the inulin, has a degree of polymerization from 2 to about 1000 and preferably from 2 to about 60, and a degree of substitution of less than 2 on the basis of one fructose unit.

According to one preferred embodiment, the hydrophobic chains contain at least one alkyl carbamate group of formula R—NH—CO—O in which R is an alkyl group containing from 4 to 32 carbon atoms or a hydrophobic ester group which is a C₄-C₃₂ alkyl ester, i.e. a —OCO—R group, R being a C₄-C₃₂ alkyl.

Advantageously, the hydrophobic ester group is a C₆-C₂₀ alkyl ester group. Preferably, the hydrophobic ester group is a C₈-C₂₀ alkyl ester group. Preferentially, the hydrophobic ester group is a C₁₀-C₂₀ alkyl ester group.

More preferentially, the hydrophobic ester group is a C₁₀-C₁₈ alkyl ester group. According to a more preferred embodiment, the hydrophobic chains are alkyl carbamate groups, in particular C₈-C₁₈ alkyl carbamate groups and more particularly lauryl carbamate groups.

These hydrophobic groups are in particular derived from the reaction of the hydroxyl groups of the starting inulin with either an isocyanate R—N═C═O (to form a carbamate group) or an acid R—COOH or acid chloride R—COCl (to form an ester group).

In particular, as non-limiting illustrations of hydrophobically modified inulins that may be used in the compositions according to the invention, mention may be made of stearoyl inulin, such as those sold under the names Lifidrem INST by the company Engelhard and Rheopearl INS by the company Ciba; palmitoyl inulin; undecylenoyl inulin, such as those sold under the names Lifidrem INUK and Lifidrem INUM by the company Engelhard; and inulin lauryl carbamate, such as the product sold under the name Inutec SP1 by the company Orafti. In particular, the hydrophobically modified fructan is an inulin grafted with lauryl carbamate, which is obtained in particular from the reaction of lauryl isocyanate with an inulin, in particular obtained from chicory. Examples of these compounds that may in particular be mentioned include the product sold under the name Inutec SP1 by the company Orafti.

The hydrophobically modified polysaccharide may be present in the composition according to the invention in a content ranging from 0.01% to 20% by weight, from 0.05% to 15% by weight, from 0.05% to 10% by weight, preferably from 0.1% to 5% by weight, better still from 0.5% to 1% by weight, relative to the total weight of the composition.

### Sulfonic Polymer

The composition according to the invention comprises a homopolymer or copolymer of a monomer comprising a sulfonic group.

The polymers comprising at least one monomer comprising a sulfonic group that are used in the composition of the invention are advantageously water-soluble or water-dispersible or water-swellable. The polymers used in accordance with the invention are homopolymers or copolymers that can be obtained from at least one ethylenically unsaturated monomer comprising a sulfonic group, which may be in free form or partially or totally neutralized form.

Preferentially, the polymers in accordance with the invention may be partially or totally neutralized with an inorganic base (sodium hydroxide, potassium hydroxide or aqueous ammonia) or an organic base such as monoethanolamine, diethanolamine or triethanolamine, an aminomethylpropanediol, N-methylglucamine, basic amino acids, for instance arginine and lysine, and mixtures of these compounds. They are generally neutralized. In the present invention, the term “neutralized” is intended to mean polymers that are totally or virtually totally neutralized, i.e. at least 90% neutralized.

The polymers used in the composition of the invention generally have a number-average molecular weight ranging from 1000 to 20 000 000 g/mol, preferably ranging from 20 000 to 5 000 000 g/mol and even more preferentially from 100 000 to 1 500 000 g/mol.

These polymers according to the invention may be crosslinked or noncrosslinked.

The monomers comprising a sulfonic group of the polymer used in the composition of the invention are in particular chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof, and mixtures thereof.

According to a preferred embodiment of the invention, the monomers comprising a sulfonic group are chosen from (meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, 2-methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid and 2-acrylamido-2,6-dimethyl-3-heptanesulfonic acid, and also partially or totally neutralized forms thereof, and mixtures thereof.

More particularly, use is made of 2-acrylamido-2-methylpropanesulfonic acid (AMPS), and also partially or totally neutralized forms thereof.

When the polymers are crosslinked, the crosslinking agents may be chosen from the polyolefinically unsaturated compounds commonly used for crosslinking polymers obtained by radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allyl or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The homopolymer of monomers comprising a sulfonic group may be crosslinked with one or more crosslinking agents.

These homopolymers are generally crosslinked and neutralized, and they may be obtained according to the preparation process comprising the following steps:

(a) the monomer such as 2-acrylamido-2-methylpropanesulfonic acid in free form is dispersed or dissolved in a solution of tert-butanol or of water and tert-butanol;

(b) the monomer solution or dispersion obtained in (a) is neutralized with one or more inorganic or organic bases, preferably aqueous ammonia NH₃, in an amount making it possible to obtain a degree of neutralization of the sulfonic acid functions of the polymer ranging from 90% to 100%;

(c) the crosslinking monomer(s) are added to the solution or dispersion obtained in (b);

(d) a standard free-radical polymerization is performed in the presence of free-radical initiators at a temperature ranging from 10 to 150° C.; the polymer precipitates in the tert-butanol-based solution or dispersion.

The preferred AMPS homopolymers are generally characterized in that they comprise, randomly distributed:

a) from 90% to 99.9% by weight of units of general formula (I) below:

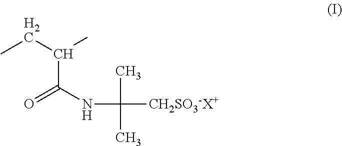

in which X⁺ denotes a proton, an alkali metal cation, an alkaline-earth metal cation or the ammonium ion, not more than 10 mol % of the cations X⁺ possibly being protons H⁺;
b) from 0.01% to 10% by weight of crosslinking units originating from at least one monomer containing at least two olefinic double bonds; the weight proportions being defined relative to the total weight of the polymer.

The homopolymers according to the invention that are more particularly preferred comprise from 98% to 99.5% by weight of units of formula (II) and from 0.2% to 2% by weight of crosslinking units.

A polymer of this type that may in particular be mentioned is the crosslinked and neutralized 2-acrylamido-2-methylpropanesulfonic acid homopolymer sold by the company Clariant under the trade name Hostacerin® AMPS (CTFA name: ammonium polyacryldimethyltauramide).

The polymer comprising sulfonic groups may be present in the composition according to the invention in an active material content ranging, for example, from 0.05% to 5% by weight, preferably ranging from 0.1% to 5% by weight, preferentially ranging from 0.1% to 2% by weight, relative to the total weight of the composition.

### Acrylic Polymer

The composition according to the invention optionally contains a (meth)acrylic acid polymer, which is preferably crosslinked.

Preferably, the (meth)acrylic acid polymer is an acrylic acid homopolymer. The polymer may be crosslinked with a crosslinking agent, in particular chosen from pentaerythritol allyl ether, sucrose allyl ether, or propylene allyl ether.

Such polymers have the INCI name: Carbomer.

Use may be made, for example, of the polymers sold by the company Lubrizol under the names Carbopol 980 or 981, or Carbopol Ultrez 10, or by the company 3V under the name Synthalen K or Synthalen L or Synthalen M.

The (meth)acrylic acid polymer may be present in the composition according to the invention in an amount ranging from 0.01% to 5% by weight, and preferably from 0.1% to 3% by weight, relative to the total weight of the composition.

### Oxyethylenated Silicone Surfactant

The compositions in accordance with the invention may comprise, as compound iii), a silicone emulsifier (or surfactant) in particular chosen from oxyethylenated polydimethylsiloxanes.

Preferably, the silicone surfactant comprises polyethylene chains on the main chain (side or pendent polyoxyethylene chains).

The number of alkylene oxide units may range from 2 to 50 and preferably from 5 to 20.

Such silicone surfactants are in particular those called PEG-10 dimethicone sold by Shin-Etsu under the name KF-6017.

The silicone surfactant may be present in the composition according to the invention in an amount ranging from 0.01% to 5% by weight, and preferably from 0.1% to 3% by weight, relative to the total weight of the composition.

Preferably, the composition according to the invention comprises at least one acrylic polymer and at least one silicone surfactant.

Preferably, the composition according to the invention also comprises at least one active agent, in particular one cosmetic active agent.

The active agents can be chosen from benzenediol derivatives, mushroom extracts, peptides and/or acylated peptides thereof, salicylic acid and salicylic acid derivatives, C-glycoside derivatives, algal extracts, and mixtures thereof.

The benzenediol derivatives are in particular the compounds of formula (II) below:

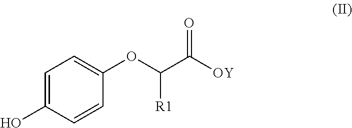

in which:
Y is chosen from H, an alkyl or alkenyl group comprising from 1 to 8 carbon atoms, a phenyl, Na⁺, K⁺ or NH₄⁺,
R1 is chosen from H, a linear or branched, saturated or unsaturated alkyl group comprising from 1 to 18 carbon atoms, a —C(═O)—R2 group, in which R2 is a linear or branched, saturated or unsaturated alkyl group comprising from 1 to 17 carbon atoms or a —(CH₂)ₙ—COOX group in which n is between 0 and 17, and X is chosen from H, an alkyl or alkenyl group comprising from 1 to 8 carbon atoms, a phenyl, Na⁺, K⁺ or NH₄⁺,
or one of its salts of organic or inorganic bases, or one of its enantiomers.

According to one particular embodiment, the soothing agent of formula (I) is characterized in that Y is chosen from the group constituted of H, methyl, ethyl, isopropyl, Na⁺ and K⁺.

According to another particular embodiment, the soothing agent of formula (I) is characterized in that R1 is chosen from the group constituted of H, a linear or branched, saturated or unsaturated alkyl group comprising from 1 to 18 carbon atoms and a —(CH₂)ₙ—COOX group in which n is 0, 1 or 2 and X is H.

According to one preferred embodiment, the soothing agent of formula (I) is characterized in that R1 is a methyl and Y is H: it is -(4-hydroxyphenoxy)propionic acid.

The term “alkyl group” is intended to mean an aliphatic saturated hydrocarbon-based group which may be linear or branched. The preferred alkyl groups comprise from 1 to 4 carbon atoms, such as methyl or ethyl. The term “alkenyl group” is understood to mean an unsaturated alkyl comprising at least one double bond between two carbon atoms.

2-(4-Hydroxyphenoxy)propionic acid, the CAS number of which is No. 67648-61-7, also known as HPPA, is a hydroquinone ether having the following expanded formula:

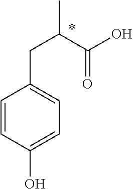

This compound is in particular sold by Sigma-Aldrich or can be obtained by preparation methods known to those skilled in the art, by organic chemistry reactions or by microbial or enzymatic hydroxylation of the phenoxy group.

The preparation of this compound is described generally in patent application EP 0 334 595. The compounds of formula (I), in particular 2-(4-hydroxyphenoxy)propionic acid are described in patent application WO 2008/107092.

2-(4-Hydroxyphenoxy)propionic acid comprises an asymmetric carbon and can therefore be in the racemic form or in the enantiomeric form R or S. In the context of the present invention, the term “2-(4-hydroxyphenoxy)propionic acid” is intended to mean 2-(4-hydroxyphenoxy)propionic acid in racemic form, or in its enantiomeric form R, that is to say (R)-(+)-2-(4-hydroxyphenoxy)propionic acid.

The 2-(4-hydroxyphenoxy)propionic acid is advantageously white in color. It is sold under the reference Radianskin PW LS 9918 by Laboratoires Sérobiologiques and/or Cognis and/or BASF.
4-(1-Phenylethyl)-1,3-benzenediol or 4-(1-phenylethyl)-1,3-dihydroxybenzene or otherwise known as phenyethyl resorcinol or phenylethylbenzenediol or else styrylresorcinol.

Such a compound is sold under the name Symwhite 377® or BIO 377 by the company Symrise.

The mushroom extracts are in particular Grifola frondosa extracts, such as the product Eterniskin LS 9881 sold by the company Laboratoires Sérobiologiques and/or Cognis and/or BASF.

A mushroom extract as previously described may be present in the composition according to the invention in an amount ranging from 0.005% to 0.5% by weight of active material (Grifola frondosa), relative to the total weight of the composition, preferably ranging from 0.01% to 0.1% by weight of active material, and preferentially ranging from 0.02% to 0.05% by weight of active material, for example about 0.03% by weight of active material.

The algal extracts are in particular Chlorella extracts.

A Chlorella extract and also the process for preparing same are as described in application FR 2 747 922. It is more particularly a natural extract of algae of the Chlorella type, more particularly a concentrated extract of Chlorella vulgaris sold under the name Dermochlorella® by the company Codif. According to one embodiment, the Chlorella extract is in the form of a solution in which it is present in an amount of 2.25% by weight relative to the total weight of the solution, the remainder being a mixture of glycerol and water, preferably in the proportions glycerol 50/water 47.75.

A Chlorella extract as previously described may be present in the composition according to the invention in an amount ranging from 0.001% to 0.5% and preferably from 0.002% to 0.25% by weight of active material (Chlorella), relative to the total weight of the composition, more particularly ranging from 0.01% to 0.1% by weight of active material, for example about 0.0225% by weight of active material.

The peptides and/or acyl peptides are in particular chosen from acyl peptides, in particular di-, tri-, tetra-, penta- and hexapeptides, more particularly from:
the N-acetyl-L-Tyrosyl-L-Prolyl-L-Phenylalanyl-L-Phenylalaninamide peptide (INCI name: ACETYL TETRAPEPTIDE-15).

Such a peptide is sold under the name Skinasensyl® PW LS 9852 by the company Laboratoire Seriobiologique.
The acetylated peptide of the hexapeptide consisting of glutamic acid-glutamic acid-methionine-glutamine-arginine-arginylamide (INCI name: ACETYL HEXAPEPTIDE-8).

Such a peptide is sold under the name Argireline® by the company Lipotec.

According to one embodiment, the peptides as previously described are present in the composition according to the invention in an amount ranging from about 0.00005% to about 0.01% by weight of active material, relative to the total weight of the composition, preferably ranging from about 0.0001% to about 0.005% by weight of active material, for example about 0.0004% by weight of active material.

Salicylic acid and salicylic acid derivatives, in particular of formula (III) below:

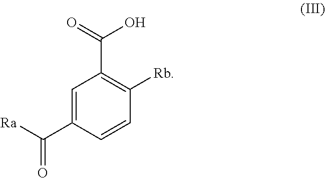

in which:
the radical Ra denotes:
   a linear, branched or cyclic, saturated aliphatic chain containing from 2 to 22 carbon atoms;
   an unsaturated chain containing from 2 to 22 carbon atoms, containing one or more double bonds which may be conjugated;
   an aromatic nucleus bonded to the carbonyl radical directly or by means of saturated or unsaturated aliphatic chains containing from 2 to 7 carbon atoms;
      it being possible for said groups to be substituted with one or more identical or different substituents chosen from:
      (a) halogen atoms,
      (b) the trifluoromethyl group,
      (c) hydroxyl groups in free form or in a form esterified with an acid containing from 1 to 6 carbon atoms, or
      (d) a carboxyl function in free form or in a form esterified with a lower alcohol containing from 1 to 6 carbon atoms;
Rb is a hydroxyl group;
   and also salts thereof derived from an inorganic or organic base.

According to one embodiment, the radical Ra denotes:
a linear, branched or cyclic, saturated aliphatic chain containing from 3 to 11 carbon atoms; or
an unsaturated chain containing from 3 to 17 carbon atoms and comprising one or more conjugated or non-conjugated double bonds; it being possible for said hydrocarbon-based chains to be substituted with one or more identical or different substituents chosen from:
   (a) halogen atoms;
   (b) the trifluoromethyl group;
   (c) hydroxyl groups in free form or in a form esterified with an acid containing from 1 to 6 carbon atoms; or
   (d) a carboxyl function in free form or in a form esterified with a lower alcohol containing from 1 to 6 carbon atoms;
   and also salts thereof obtained by salification with an inorganic or organic base.

The compounds that are more particularly preferred are those in which the radical Ra is a C₃-C₁₁ alkyl group. Among the compounds of formula (III) that are particularly preferred, mention may be made of: 5-n-octanoylsalicylic acid (or capryloylsalicylic acid); 5-n-decanoylsalicylic acid; 5-n-dodecanoylsalicylic acid; 5-n-heptyloxysalicylic acid, and the corresponding salts thereof.

The salicylic acid compound is advantageously chosen from salicylic acid and 5-n-octanoylsalicylic acid. 5-n-Octanoylsalicylic acid will more particularly be used. 5-n-Octanoylsalicyliic acid (or capryloylsalicylic acid) is provided under the name Mexoryl SAB® by the company Chimex.

Examples of inorganic bases that may be mentioned include alkali metal or alkaline-earth metal hydroxides, for instance sodium hydroxide, potassium hydroxide or aqueous ammonia. Among the organic bases that may be mentioned are amines and alkanolamines. Quaternary salts, for instance those described in patent FR 2 607 498, are particularly advantageous. The compounds of formula (III) that may be used according to the invention are described in patents U.S. Pat. No. 6,159,479, U.S. Pat. No. 5,558,871, FR 2 581 542, FR 2 607 498, U.S. Pat. No. 4,767,750, EP 378 936, U.S. Pat. No. 5,267,407, U.S. Pat. No. 5,667,789, U.S. Pat. No. 5,580,549 and EP A-570 230.

According to one embodiment, the salicylic acid or the salicylic acid compound of formula (III) as previously described is present in the composition according to the invention in an amount ranging from about 0.1% to about 5% by weight of active material (salicylic acid or derivative), relative to the total weight of the composition, preferably ranging from about 0.1% to about 1% by weight of active material, for example about 0.3% by weight of active material.

The C-Glycoside Derivatives are in Particular of Formula (IV):

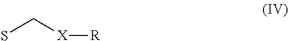

in which:
R denotes an unsubstituted linear C₁-C₄ alkyl radical, in particular C₁-C₂ alkyl radical, in particular methyl;
S represents a monosaccharide chosen from D-glucose, D-xylose, N-acetyl-D-glucosamine and L-fucose, and in particular D-xylose;
X represents a group chosen from —CO—, —CH(OH)— and —CH(NH₂)—, and preferentially a —CH(OH)— group.

As nonlimiting illustrations of C-glycoside derivatives that are more particularly suitable for use in the invention, mention may be made in particular of the following derivatives:
C-beta-D-xylopyranoside-n-propan-2-one;
C-alpha-D-xylopyranoside-n-propan-2-one;
C-beta-D-xylopyranoside-2-hydroxypropane;
C-alpha-D-xylopyranoside-2-hydroxypropane;
1-(C-beta-D-glucopyranosyl)-2-hydroxypropane,
1-(C-alpha-D-glucopyranosyl)-2-hydroxypropane;
1-(C-beta-D-glucopyranosyl)-2-aminopropane;
1-(C-alpha-D-glucopyranosyl)-2-aminopropane;
3′-(acetamido-C-beta-D-glucopyranosyl)propan-2′-one;
3′-(acetamido-C-alpha-D-glucopyranosyl)propan-2′-one;
1-(acetamido-C-beta-D-glucopyranosyl)-2-hydroxypropane,
1-(acetamido-C-beta-D-glucopyranosyl)-2-aminopropane;
isomers thereof and mixtures thereof.

According to one embodiment, C-beta-D-xylopyranoside-2-hydroxypropane or C-alpha-D-xylopyranoside-2-hydroxypropane, and better still C-beta-D-xylopyranoside-2-hydroxypropane, may be advantageously used for the preparation of a composition according to the invention.

According to one particular embodiment, the C-glycoside derivative (IV) is C-3-D-xylopyranoside-2-hydroxypropane in the form of a solution containing 30% by weight of active material in a water/propylene glycol mixture (60/40% by weight) such as the product manufactured by Chimex under the trade name Mexoryl SBB®.

A C-glycoside derivative (IV) is in particular described in document WO 02/051828.

According to one embodiment, the composition according to the invention comprises a C-glycoside derivative in a proportion of from about 0.1% to about 15% by weight of active material (C-glycoside derivative) relative to the total weight of the composition, in particular from about 0.5% to about 10% by weight of active material relative to the total weight of the composition, more particularly from about 1% to about 7.5% by weight of active material relative to the total weight of the composition, and more particularly from about 1.5% to about 5% by weight of active material, for example about 3% by weight of active material.

Preferably, the composition according to the invention may also comprise pigments and/or nacres and/or fillers that are normally used in cosmetic compositions.

The term “pigments” should be understood to mean white or colored, mineral or organic particles of any shape, which are insoluble in the physiological medium, and which are intended to color the composition.

The term “fillers” should be understood to mean colorless or white, mineral or synthetic, lamellar or non-lamellar particles, which are intended to give body or rigidity to the composition, and/or immediate effects, such as softness, matting, coverage, a powdered finish and uniformity, to the makeup.

The term “nacres” should be understood to mean iridescent particles of any form, in particular produced by certain molluscs in their shell, or else synthesized.

The pigments may be present in the composition in a proportion of from 0.01% to 25% by weight of the final composition, and preferably in a proportion of from 0.1% to 10% by weight. These pigments may be white or colored, and mineral or organic. Mention may be made of titanium, zirconium or cerium oxides, and also zinc, iron or chromium oxides, ferric blue, chromium hydrate, carbon black, ultramarines (aluminosilicate polysulfides), manganese pyrophosphate and certain metal powders such as silver or aluminum powders. Mention may also be made of D&C pigments and lakes, commonly used to give the lips and the skin a makeup effect, which are calcium, barium, aluminum, strontium or zirconium salts.

The nacres may be present in the composition in a proportion of from 0.01% to 20% by weight, preferably in a content of about from 0.1% to 10% by weight. Among the nacres that can be envisioned, mention may be made of natural nacre, mica coated with titanium oxide, with iron oxide, with natural pigment or with bismuth oxychloride, and also colored titanium mica.

The composition according to the invention may also comprise one or more fillers, in particular in a content ranging from 0.01% to 50% by weight and preferably ranging from 0.02% to 10% by weight relative to the total weight of the composition. The fillers may be mineral or organic of any shape, platelet-shaped, spherical or oblong. Mention may be made of talc, mica, silica, kaolin, polyamide powder (Nylon®), poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), or of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

According to one embodiment, the present invention relates to a cosmetic composition comprising, in addition to the compounds i), ii) and iii):
at least one C-glucoside derivative of formula (I);
at least one benzenediol derivative;
at least salicylic acid or a compound of formula (III);
at least one peptide and/or one acylpeptide;
at least one mushroom extract;
at least one algal extract.

The composition according to the invention comprises a physiologically acceptable aqueous medium.

The term “physiologically acceptable medium” is intended to denote a medium that is compatible with human keratin materials and/or fibers, for instance, in a non-limiting manner, the skin, the mucous membranes, the nails, the scalp and/or the hair.

This physiologically acceptable aqueous medium comprises water, optionally as a mixture, or not, with one or more organic solvents, such as a C₁-C₈ alcohol, in particular ethanol, isopropanol, tert-butanol, n-butanol, polyols such as glycerol, propylene glycol, butylene glycol, and polyol ethers.

A composition according to the invention may also comprise a fatty phase, which may comprise oils, gums and waxes normally used in the field of application under consideration.

Thus, according to one embodiment, a composition according to the invention may also comprise at least one fatty phase chosen from a fatty phase which is solid at ambient temperature (20-25° C.) and atmospheric pressure and/or a fatty phase which is liquid at ambient temperature (20-25° C.) and atmospheric pressure.

A liquid fatty phase suitable for implementing the invention may comprise a volatile oil, a non-volatile oil, and a mixture thereof. A volatile or non-volatile oil may be a hydrocarbon oil, in particular of animal or vegetable origin, a synthetic oil, a silicone oil, a fluorinated oil or a mixture thereof.

A solid fatty phase suitable for implementing the invention may be, for example, chosen from pasty fatty substances, gums, and mixtures thereof.

As oils that can be used in the invention, mention may be made of mineral oils (for example liquid petroleum jelly), vegetable oils (for example liquid fraction of shea butter, sunflower oil), synthetic oils (Purcellin oil), fatty acid esters, silicone oils or waxes and fluorinated oils (perfluoropolyethers). Fatty alcohols and fatty acids may be added to these oils.

When a composition is an emulsion, the proportion of the fatty phase may range from 5% to 80% by weight and preferably from 5% to 50% by weight relative to the total weight of the composition. The oils, waxes, emulsifiers and co-emulsifiers used in the composition in emulsion form are chosen from those conventionally used in the cosmetics field.

One or more emulsifiers may be present in a composition of the invention in a proportion ranging from 0.3% to 30% by weight and in particular from 0.5% to 20% by weight relative to the total weight of the composition.

The composition according to the invention may also contain cosmetic adjuvants in particular chosen from emulsifiers, gelling agents, oils, waxes, preserving agents, antioxidants, water, fragrances, fillers, UV screens, pigments, fibers, chelating agents, odor absorbers and colorants.

The amounts of these various adjuvants are those conventionally used in the cosmetics field, and may range, for example, from 0.01% to 30% of the total weight of the composition. In general, the amounts are adjusted as a function of the formulation prepared. Depending on their nature, these adjuvants may be introduced into the fatty phase, into the aqueous phase and/or into the lipid spherules.

The composition according to the invention may be in the form of an aqueous or aqueous-alcoholic solution; a dispersion; a water-in-oil, oil-in-water or multiple emulsion; a suspension; microcapsules or microparticles; vesicular dispersions of ionic and/or non-ionic type; or an aerosol composition also comprising a pressurized propellant. Preferentially, the composition according to the invention may be an oil-in-water or water-in-oil emulsion. More preferentially, the composition according to the invention is an oil-in-water emulsion. When the composition comprises an oily phase, the latter may comprise one or more silicone elastomers. Examples of silicone elastomers are described in application WO-A-2009/080958. The silicone elastomers are in particular the products sold under the name KSG by the company Shin-Etsu, under the names Trefil, BY29 or EPSX by the company Dow Corning or under the name Gransil by the company Grant Industries.

A composition according to the invention may be in the form of a care product, an antisun or after-sun product, a daily photoprotective care product, a body product, a foundation to be applied to the face or the neck, a concealer product, a complexion corrector, a tinted cream or a makeup base for making up the face, or a body makeup composition.

A composition according to the invention may be used for the purposes of improving the general condition of the epidermis, in particular of the skin, and especially for maintaining or restoring its physiological functions and/or its aesthetic appearance.

The viscosity of a composition of the invention may be measured via any method known to those skilled in the art, and in particular according to the following conventional method. Thus, the measurement can be carried out at 25° C. using a Rheomat 180 viscometer equipped with a spindle rotating at 200 rpm. Those skilled in the art may select the spindle for measuring the viscosity from the spindles M1, M2, M3 and M4 on the basis of their general knowledge, so as to be able to perform the measurement.

Other characteristics and advantages of the invention will emerge more clearly from the examples that follow, which are given as non-limiting illustrations. In the text hereinbelow or hereinabove, the proportions are given as weight percentages, unless otherwise indicated.

### EXAMPLES 1 TO 3

Two compositions (ex 1 and 2) according to the invention and one composition (ex 3) that is similar but contains neither acrylic polymer nor silicone surfactant were prepared.

The viscosity of the compositions obtained was then measured after 24 hours of storage at ambient temperature (viscosity measured at 25° C. using a Rheomat 180 viscometer with an M3 spindle after 10 minutes of rotation at 200 rpm). The following results were obtained:

| | Example (in % AM) | | |
|---|---|---|---|
| | 1 | 2 | |
| | (Invention) | (Invention) | 3 |
| 5-n-Octanoylsalicylic acid | 0.3 | 0.3 | 0.3 |
| Polyacrylamidomethylpropanesulfonic | 0.78 | 0.78 | 0.78 |
| acid partially neutralized with | | | |
| aqueous ammonia and crosslinked | | | |
| (Hostacerin AMPS ® from Clariant) | | | |
| Inulin lauryl carbamate (Inutec SP1 | 0.73 | 0.73 | 0.73 |
| from Beneo) | | | |
| Ammonium | | 0.27 | |
| acryloyldimethyltaurate/steareth-8 | | | |
| methacrylate copolymer (Aristoflex | | | |
| SNC from Clariant) | | | |
| Crosslinked acrylic acid | 0.4 | | |
| homopolymer (Carbopol Ultrez 10 | | | |
| from Lubrizol) | | | |
| PEG-10 Dimethicone | 1.3 | 1.3 | |
| Mixture of silicone elastomers and | 15 | 15 | 15 |
| cyclohexasiloxane (13/87) (Gransil | | | |
| RPS-D6 from Grant Industries) | | | |
| Squalane | 3 | 3 | 3 |
| Disodium salt of | 0.1 | 0.1 | 0.1 |
| ethylenediaminetetraacetic acid | | | |
| EDTA | | | |
| Hydroxyethylcellulose (Natrosol | 0.15 | 0.15 | 0.15 |
| 250 HHR from Aqualon) | | | |
| Preserving agent | qs | qs | qs |
| Water | qs 100% | qs 100% | qs 100% |
| microscopic appearance at T = 24 h | OK | OK | NOT OK |
| | Fine and | Fine and | Coarse |
| | regular | regular | emulsion with |
| | emulsion | emulsion | large oil |
| | with clear | with clear | globules and |
| | edges | edges | untidy edges |
| microscopic appearance after 2 | OK | OK | NOT OK |
| months at 25° C. | | | Coarse |
| | | | emulsion with |
| | | | large |
| | | | globules and |
| | | | untidy edges |

Results of the same type were attained when the 5-n-octanoylsalicylic acid was replaced with one of the following compounds:

2-(4-Hydroxyphenoxy)propionic acid (RADIANSKIN PW LS 9918) 1% AM (MP at 100% AM)
HYDROXYPROPYL TETRAHYDROPYRANTRIOL at 35% of active material in a 60/40% water/propylene glycol mixture: 3.15% AM GRIFOLA FRONDOSA FRUITING BODY EXTRACT (ETERNISKIN LS9881) 0.03% AM

### ACETYL TETRAPEPTIDE-15 (SKINASENSYL) 0.00042% AM

### CHLORELLA VULGARIS HYDROGLYCEROL EXTRACT

### (DERMOCHLORELLA) 0.0275% AM

MICROSPHERES (VINYLIDENE CHLORIDE/ACRYLONITRILE/METHYL METHACRYLATE COPOLYMER) EXPANDED BY ISOBUTANE (EXPANCEL 551 DE 40 D42 from AKZO NOBEL), 0.1% AM
ANATASE TITANIUM OXIDE COATED WITH ALUMINUM STEAROYL GLUTAMATE (NAI-TAO-77891 from MYOSHI KASEI), 0.25%
MICA (and) BISMUTH OXYCHLORIDE (and) CARMINE (CHROMA-LITE MAGENTA CL4505 from BASF), 0.5%
Iron oxide-coated mica (PEARLESCENT PIGMENT PRESTIGE SOFT COPPER from ECKART): 0.3%

These tests show that the composition comprising the combination of sulfonic polymer (Hostacerin AMPS), of hydrophobic polysaccharide, of crosslinked acrylic polymer (carbomer such as Ultrez 10) and of oxyethylenated silicone surfactant makes it possible to obtain stable compositions comprising an active agent and/or a particular compound.

These compositions are stable for at least 2 months at 45° C.

1. A composition, comprising, in a physiologically acceptable aqueous medium:
i) at least one polymer comprising a sulfonic group;
ii) at least one polysaccharide modified with hydrophobic chains; and
iii) at least one (meth)acrylic acid polymer, silicone surfactant which is an oxyethylenated polydimethylsiloxane, or both.

2. The composition of claim 1, wherein the polysaccharide is a fructan comprising hydrophobic chains.

3. The composition of claim 1, wherein the hydrophobic chains of the polysaccharide are
linear or branched, saturated or unsaturated hydrocarbon-based chains containing from 1 to 50 carbon atoms;
divalent cycloaliphatic groups or organopolysiloxane chains comprising one or more ester, amide, urethane, carbamate, thiocarbamate, urea, thiourea and/or sulfonamide functions; or
divalent aromatic groups.

4. The composition of claim 2, wherein the fructan comprises inulin having a degree of polymerization from 2 to about 1000 and a degree of substitution of less than 2 on the basis of one fructose unit.

5. The composition of claim 1, wherein the hydrophobic chains comprise at least one alkyl carbamate group of formula R—NH—CO— in which R is an alkyl group comprising from 1 to 22 carbon atoms.

6. The composition of claim 1, wherein the hydrophobic chains are lauryl carbamate groups.

7. The composition of claim 1, wherein the polysaccharide is present in a content ranging from 0.01% to 20% by weight, relative to the total weight of the composition.

8. The composition of claim 1, wherein the polymer comprising a sulfonic group is a 2-acrylamido-2-methylpropanesulfonic acid polymer.

9. The composition of claim 1, wherein the polymer comprising a sulfonic group is a crosslinked 2-acrylamido-2-methylpropanesulfonic acid homopolymer.

10. The composition of claim 1, wherein the polymer comprising sulfonic groups is present in a content ranging from 0.05% to 5% by weight, relative to the total weight of the composition.

11. The composition of claim 1, comprising the (meth)acrylic acid polymer which is an acrylic acid homopolymer.

12. The composition of claim 1, comprising an acrylic acid polymer is present in an amount ranging from 0.01% to 5% by weight relative to the total weight of the composition.

13. The composition of claim 1, comprising a silicone surfactant comprising from 2 to 50 mol of ethylene oxide.

14. The composition of claim 1, comprising the silicone surfactant in an amount ranging from 0.01% to 5% by weight relative to the total weight of the composition.

15. The composition of claim 1, comprising:
i) the at least one polymer comprising a sulfonic group;
ii) the at least one polysaccharide modified with hydrophobic chains;
iii) the at least one (meth)acrylic acid polymer; and
iv) the at least one silicone surfactant which is an oxyethylenated polydimethylsiloxane.

16. The composition of claim 1, further comprising at least one active agent, pigment, nacre, filler, or mixture thereof.

17. The composition of claim 1, further comprising an active agent selected from the group consisting of a benzenediol derivative, a derivative, a mushroom extract, a peptide, salicylic, a salicylic acid derivative, a C-glycoside derivative, an algal extract, and a mixture thereof.

18. The composition of claim 1, further comprising a benzenediol derivative active agent which is a compound of formula (II): 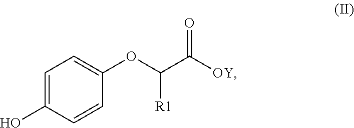
wherein:
Y is H, an alkyl or alkenyl group comprising from 1 to 8 carbon atoms, a phenyl, Na⁺, K⁺ or NH₄⁺,
R1 is from H, a linear or branched, saturated or unsaturated alkyl group comprising from 1 to 18 carbon atoms, a —C(═O)—R2 group, in which R2 is a linear or branched, saturated or unsaturated alkyl group comprising from 1 to 17 carbon atoms or a —(CH₂)ₙ—COOX group in which n is between 0 and 17, and X is chosen from H, an alkyl or alkenyl group comprising from 1 to 8 carbon atoms, a phenyl, Na⁺, K⁺ or NH₄⁺,
or one of its salts of organic or inorganic bases, or one of its enantiomers.

19. The composition of claim 18, wherein R1 is a methyl and Y is H.

20. The composition of claim 1, further comprising a C-glycoside derivative active agent having formula (IV): 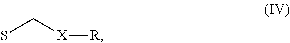
wherein:
R denotes an unsubstituted linear C₁-C₄ alkyl radical;
S represents a monosaccharide chosen from D-glucose, D-xylose, N-acetyl-D-glucosamine and L-fucose;
X represents a group chosen from —CO—, —CH(OH)—, —CH(NH₂)—; and
isomers thereof and mixtures thereof.

21. The composition of claim 1, further comprising a salicylic acid derivative active agent having formula (III): 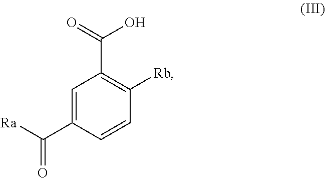
wherein:
the radical Ra denotes:
a linear, branched or cyclic, saturated aliphatic chain containing from 2 to 22 carbon atoms;
an unsaturated chain containing from 2 to 22 carbon atoms, containing one or more double bonds which may be conjugated;
an aromatic nucleus bonded to the carbonyl radical directly or by saturated or unsaturated aliphatic chains containing from 2 to 7 carbon atoms;
it being possible for said groups to be substituted with one or more identical or different substituents chosen from:
(i) halogen atoms,
(ii) trifluoromethyl group,
(iii) hydroxyl groups in free form or in a form esterified with an acid containing from 1 to 6 carbon atoms, or
(iv) a carboxyl function in free form or in a form esterified with a lower alcohol containing from 1 to 6 carbon atoms;
Rb is a hydroxyl group;
or a salt thereof derived from an inorganic or organic base.

22. The composition of claim 1, further comprising a mushroom extract active agent which is a Grifola frondosa extract.

23. The composition of claim 1, further comprising a peptide active agent.

24. The composition of claim 1, which is in the form of an oil-in-water emulsion.

25. The composition of claim 1, further comprising a cosmetic additive selected from the group consisting of an emulsifier, a gelling agent, an oil, a wax, a preserving agent, an antioxidant, water, a fragrance, a UV screen, a fiber, a chelating agent, an odor absorber, a colorant and a mixture thereof.

26. A non-therapeutic cosmetic treatment process, comprising applying the composition of claim 1 to a keratin material.
